(19) United States
(12) Patent Application Publication
(10) Pub. No.: US 2021/0228695 A1
(43) Pub. Date: Jul. 29, 2021

(54) PIC1 PEPTIDE COMPOSITIONS AND METHODS OF USE THEREOF FOR TREATMENT OF DRUG-INDUCED IMMUNE HEMOLYTIC ANEMIA

(71) Applicant: REALTA HOLDINGS, LLC, Norfolk, VA (US)

(72) Inventors: Neel K. KRISHNA, Norfolk, VA (US); Kenji CUNNION, Norfolk, VA (US)

(73) Assignee: REALTA HOLDINGS, LLC, Norfolk, VA (US)

(21) Appl. No.: 16/972,340

(22) PCT Filed: Jun. 5, 2019
(86) PCT No.: PCT/US2019/035503

§ 371 Date: Dec. 4, 2020

Related U.S. Application Data
(60) Provisional application No. 62/681,478, filed on Jun. 6, 2018.

(51) Int. Cl. A61K 38/57; A61K 47/60; A61P 7/06

(52) U.S. Cl. CPC A61K 38/57; A61P 7/06; A61K 47/60

(57) ABSTRACT

Compositions and methods for treating hemolytic anemia are described. A classical complement pathway inhibitor is used to treat hemolytic anemia, for example ceftriaxone-induced complement-mediated hemolysis. A Complement Hemolysis Using Human Erythrocytes (CHUHE) assay is also described where exogenous ceftriaxone is added to a patients serum to show enhanced lysis of the patients erythrocytes in vitro. Ceftriaxone is shown to initiate classical complement pathway-mediated hemolysis by ex vivo reversal with Peptide Inhibitor of Complement C1 (PIC1).

### CROSS-REFERENCE TO RELATED APPLICATIONS

This application claims priority to U.S. Provisional Application No. 62/681,478, filed Jun. 6, 2018, the disclosure of which is herein incorporated by reference in its entirety.

### BACKGROUND

Anemia is a condition where a decrease in the number of red blood cells (RBCs) or hemoglobin results in a diminished ability of the blood to carry oxygen. Symptoms of anemia generally include fatigue, lack of energy, lightheadedness or dizziness, especially when sitting up rapidly, or standing, shortness of breath, headaches, a pale appearance, rapid heart rate or palpitations, and chest pain. Acquired hemolytic anemia is a type of anemia that arises from hemolysis of blood cells at a higher rate than blood cell formation of such blood cells. Some forms of acquired hemolytic anemia can be induced by drugs.

Ceftriaxone is the second most common agent to cause drug-induced hemolytic anemia. Ceftriaxone-induced immune hemolytic anemia (CIIHA) is a rare complication that can lead to shock, multi-organ dysfunction and death. Most documented cases of CIIHA have occurred in children. Cases of CIIHA are primarily reported in patients with an underlying condition of sickle cell disease or HIV, with a fatality rate of 30% in published cases. Because CIIHA is a type 2 hypersensitivity reaction, the disease process can progress rapidly after re-exposure.

There is a need for new treatments for drug-induced hemolytic anemia, such as CIIHA.

### BRIEF DESCRIPTION OF THE DRAWINGS

FIG. 1A shows the blood (erythrocyte) hemoglobin level in a patient over 72 hours of hospitalization and timing of pRBC transfusions. The patient is a 10-year-old female with chronic active Epstein-Barr virus (EBV) disease and hemophagocytic lymphohistiocytosis (HLH). After the patient was treated was treated with ceftriaxone, the patient developed ceftriaxone-induced immune hemolytic anemia (CIIHA).

FIG. 1B shows the results of a modified assay of complement hemolysis using human erythrocytes (CHUHE) that tested the patient's serum and erythrocytes. Addition of exogenous ceftriaxone increased hemolysis over baseline (p=0.02). Addition of PIC1 (0.75 mM) to the patient's serum, to which ceftriaxone was added, resulted in decreased hemolysis compared with the addition of ceftriaxone alone (p=0.03). Means±SD are shown for 2 independent experiments.

### SUMMARY OF THE INVENTION

In one aspect is provided a method of treating hemolytic anemia in a subject comprising administering a classical complement pathway inhibitor to the subject. In some embodiments, the hemolytic anemia is induced by a drug. In some embodiments, the drug is an antibiotic. In some embodiments, the antibiotic is cefotetan, ceftriaxone, or piperacillin. In a specific embodiment, the drug is ceftriaxone.

In various embodiments, the classical complement pathway inhibitor is PIC1. In some embodiments, the PIC1 comprises one or more PEG moieties. In some embodiments, the PIC1 comprises at least about 90% sequence identity to at least one amino acid sequence selected from the group consisting of SEQ ID NO: 3-47. In a specific embodiment, the PIC1 is PA-dPEG24. In a specific embodiment, the PA-dPEG24 comprises the sequence of IALILEPICCQERAA-dPEG24 (SEQ ID NO: 19).

In some embodiments, the classical complement pathway inhibitor is administered once per day. In some embodiments, the classical complement pathway inhibitor is administered up to four times per day. In some embodiments, the classical complement pathway inhibitor is administered for 1, 2, 3, 4, 5, or 6 days.

### DETAILED DESCRIPTION

Described herein are compositions and methods for treating hemolytic anemia, such as hemolytic anemia caused by antibiotics, cancer or cancer treatments.

Unless otherwise specified, all terms used in disclosing the invention, including technical and scientific terms, have the meaning as commonly understood by one of ordinary skill in the art to which this invention belongs. By means of further guidance, term definitions may be included to better appreciate the teaching of the present invention. When certain terms are explained or defined in connection with a particular aspect or embodiment, such connotation is meant to apply throughout this specification, i.e., also for other aspects or embodiments, unless otherwise specified or unless the context clearly dictates otherwise.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The term also encompasses “consisting of” and “consisting essentially of”.

The recitation of numerical ranges by endpoints includes all numbers and fractions subsumed within the respective ranges, as well as the recited endpoints.

The term “about” as used herein when referring to a measurable value such as a parameter, an amount, a temporal duration, and the like, is meant to encompass variations of and from the specified value, in particular variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

As used herein, the terms “treat” or “treatment” refer to both therapeutic treatment and prophylactic or preventative measures, wherein the object is to prevent or slow down (lessen) an undesired physiological change or disorder, such as the development of a neurological disorder. Beneficial or desired clinical results include, but are not limited to, prevention of a disorder, reducing the incidence of a disorder, alleviation of symptoms associated with a disorder, diminishment of extent of a disorder, stabilizing (i.e., not worsening) the state of a disorder, delaying or slowing progression of a disorder, amelioration or palliation of the state of a disorder, remission (whether partial or total), whether detectable or undetectable, or combinations thereof “Treatment” can also mean prolonging survival as compared to expected survival if not receiving treatment.

Except when noted, “subject” or “patient” are used interchangeably and refer to animals, preferably warm-blooded animals, more preferably vertebrates, even more preferably mammals, still more preferably primates, and specifically include human patients and non-human mammals and primates. “Mammalian” subjects refer to any animal classified as such and include, but are not limited to, humans, domestic animals, commercial animals, farm animals, zoo animals, sport animals, pet and experimental animals such as dogs, cats, guinea pigs, rabbits, rats, mice, horses, cattle, cows, wolves, lions, tigers, horses, donkeys, zebras, cows, pigs, sheep, deer, giraffes, and rodents such as mice, rats, hamsters and guinea pigs. Preferred subjects are human patients.

In one aspect is provided a method of treating hemolytic anemia in a subject comprising administering a therapeutically effective amount of a classical complement pathway inhibitor to the subject. In yet another related aspect is provided a classical complement pathway inhibitor for the treatment of hemolytic anemia. In some embodiments, the hemolytic anemia is induced by a drug. In some embodiments, the drug is an antibiotic. In some embodiments, the antibiotic is cefotetan, ceftriaxone, or piperacillin. In a specific embodiment, the drug is ceftriaxone.

In a related aspect is provided a method of treating hemolytic anemia in a subject comprising administering a therapeutically effective amount of a classical complement pathway inhibitor to the subject.

Hemolytic anemia may be characterized by one or more of elevated serum bilirubin, excess urinary urobilinogen, reduced plasma haptoglobin, raised serum lactic dehydrogenase (LDH), hemosiderinuria, methemalbuminemia, spherocytosis, reticulocytosis, and/or erythroid hyperplasia of the bone marrow. In some embodiments, the methods for treating hemolytic anemia are effective to reduce the level of serum bilirubin in the patient as compared to the level in the patient before administration of the classical complement pathway inhibitor. In some embodiments, the methods for treating hemolytic anemia are effective to reduce the level of urinary urobilinogen in the patient as compared to the level in the patient before administration of the classical complement pathway inhibitor. In some embodiments, the methods for treating hemolytic anemia are effective to increase the level of plasma haptoglobin in the patient as compared to the level in the patient before administration of the classical complement pathway inhibitor. In some embodiments, the methods for treating hemolytic anemia are effective to reduce the level of LDH in the patient as compared to the level in the patient before administration of the classical complement pathway inhibitor.

In various embodiments, the hemolytic anemia is drug-induced. In various embodiments, the hemolytic anemia is induced by an antibiotic, e.g., cefotetan, ceftriaxone, or piperacillin.

Cefotetan (sold under trade names Apatef and Cefotan) is a cephamycin antibiotic that can be administered intravenously or intramuscularly. Cefotetan is highly resistant to a broad spectrum of beta-lactamases and is active against a wide range of both aerobic and anaerobic Gram-positive and Gram-negative microorganisms. Ceftriaxone (sold under trade name Rocephin) is a third-generation antibiotic from the cephalosporin family of antibiotics. Ceftriaxone is used to against organisms that tend to be resistant to many other antibiotics. Ceftriaxone may be administered intravenously or intramuscularly. Piperacillin is a broad-spectrum β-lactam antibiotic of the ureidopenicillin class that is used almost exclusively in combination with tazobactam for the treatment of hospital-acquired infections (such as those from Pseudomonas aeruginosa).

Other drugs that may induce hemolytic anemia include, but are not limited to, carboplatin, cefamandole, cefazolin, cefixime, cefoxitin, ceftazidime, ceftizoxime, cefuroxime, cephalexin, cephalosporins, cephalothin, chlorpropamide, cimetidine, dapsone, diclofenac, erythromycin, fludarabine, hydrochlorothiazide, levodopa, levofloxacin, mefloquine, methyldopa, nafcillin, nitrofurantoin, nonsteroidal anti-inflammatory drugs, oxaliplatin, penicillin (and its derivatives), phenacetin, phenazopyridine (pyridium), probenecid, procainamide, quinidine, rifampin, sulfamethoxazole, ticarcillin, tolectin, trimethoprim, and β-lactamase inhibitors.

In various embodiments, the patient with drug-induced hemolytic anemia is a child. Without wishing to be bound by theory, the majority of documented cases of drug-induced hemolytic anemia (e.g., ceftriaxone-induced immune hemolytic anemia (CIIHA)) have occurred in children. The mechanism for drug-induced hemolysis may involve complement activation due to the formation of drug-dependent anti-ceftriaxone antibodies. The subject may have an underlying condition, such as sickle cell disease, Epstein-Barr virus (EBV) disease, hemophagocytic lymphohistiocytosis (HLH), and HIV. If the drug-induced hemolytic anemia is a type 2 hypersensitivity reaction, the disease process can progress rapidly after re-exposure to the drug.

Without wishing to be bound by theory, CIIHA is mediated by anti-ceftriaxone antibodies that bind to the circulating ceftriaxone so as to create immune complexes that initiate classical complement pathway activation, which leads to lysis of erythrocytes. While anti-ceftriaxone antibodies develop in 12.5% of patients frequently exposed to ceftriaxone, CIIHA is a very rare complication. The standard evaluation for suspected CIIHA includes DAT testing and evaluation for the presence of anti-ceftriaxone antibodies. A diagnosis of CIIHA is inferred if the DAT is C3+ and the presence of anti-ceftriaxone antibodies is confirmed.

The standard evaluation for CIIHA does not show whether the anti-ceftriaxone antibodies are interacting with ceftriaxone to initiate classical complement pathway-mediated hemolysis. Described herein is a novel assay based on an assay of complement hemolysis using human erythrocytes (CHUHE) with PIC1. The CHUHE assay utilizes human serum and human erythrocytes to measure complement-mediated hemolysis for the specific serum and specific erythrocytes that are co-incubated.

Also provided is the use of PIC1 for the treatment of drug-induced hemolytic anemia. Also provided is the use of PIC1 for the treatment of antibiotic-induced immune hemolytic anemia. Further provided is the use of PIC1 for the treatment of ceftriaxone-induced immune hemolytic anemia (CIIHA).

“PIC1” refers to a peptide comprising the polar assortant (PA) sequence of IALILEPICCQERAA (SEQ ID NO: 1), as well as peptides comprising the same amino acid sequence but with modifications such as PEGylation. In various embodiments of the methods and compositions described herein, the PIC1 peptide comprises the amino acid sequences and modifications as set forth in SEQ ID NOs: 1-45 that are listed in Table 1.

TABLE 1

| Peptide name | Peptide sequence |
|---|---|
| PA | IALILEPICCQERAA (SEQ ID NO: 1) |
| PA-I1Sar | (Sar)ALILEPICCQERAA (SEQ ID NO: 2) |
| PA-A2Sar | I(Sar)LILEPICCQERAA (SEQ ID NO: 3) |
| PA-L3Sar | IA(Sar)ILEPICCQERAA (SEQ ID NO: 4) |
| PA-I4Sar | IAL(Sar)LEPICCQERAA (SEQ ID NO: 5) |
| PA-L5Sar | IALI(Sar)EPICCQERAA (SEQ ID NO: 6) |
| PA-E6Sar | IALIL(Sar)PICCQERAA (SEQ ID NO: 7) |
| PA-P7Sar | IALILE(Sar)ICCQERAA (SEQ ID NO: 8) |
| PA-I8Sar | IALILEP(Sar)CCQERAA (SEQ ID NO: 9) |
| PA-C9Sar | IALILEPI(Sar)CQERAA (SEQ ID NO: 10) |
| PA-C10Sar | IALILEPIC(Sar)QERAA (SEQ ID NO: 11) |
| PA-Q11Sar | ALILEPICC(Sar)ERAA (SEQ ID NO: 12) |
| PA-E12Sar | IALILEPICCQ(Sar)RAA (SEQ ID NO: 13) |
| PA-R13Sar | IALILEPICCQE(Sar)AA (SEQ ID NO: 14) |
| PA-A14Sar | IALILEPICCQER(Sar)A (SEQ ID NO: 15) |
| PA-A15Sar | IALILEPICCQERA(Sar) (SEQ ID NO: 16) |
| dPEG24-PA-dPEG24 | dPEG24-IALILEPICCQERAA-dPEG24 (SEQ ID |
| | NO: 17) |
| dPEG24-PA | dPEG24-IALILEPICCQERAA (SEQ ID NO: 18) |
| PA-dPEG24 | IALILEPICCQERAA-dPEG24 (SEQ ID NO: 19) |
| PA-dPEG20 | IALILEPICCQERAA-dPEG20 (SEQ ID NO: 20) |
| PA-dPEG16 | IALILEPICCQERAA-dPEG16 (SEQ ID NO: 21) |
| PA-dPEG12 | IALILEPICCQERAA-dPEG12 (SEQ ID NO: 22) |
| PA-dPEG08 | IALILEPICCQERAA-dPEG08 (SEQ ID NO: 23) |
| PA-dPEG06 | IALILEPICCQERAA-dPEG06 (SEQ ID NO: 24) |
| PA-dPEG04 | IALILEPICCQERAA-dPEG04 (SEQ ID NO: 25) |
| PA-dPEG03 | IALILEPICCQERAA-dPEG03 (SEQ ID NO: 26) |
| PA-dPEG02 | IALILEPICCQERAA-dPEG02 (SEQ ID NO: 27) |
| PA-C9SarC10A | IALILEPI(Sar)AQERAA (SEQ ID NO: 28) |
| PA-C9SarA10 | IALILEPI(Sar)QERAA (SEQ ID NO: 29) |
| PA-P7SarC9Sar | IALILE(Sar)I(Sar)CQERAA (SEQ ID NO: 30) |
| PA-E6Sar-dPEG24 | IALIL(Sar)PICCQERAA-dPEG24 (SEQ ID NO: |
| | 31) |
| PA-Q11Sar-dPEG24 | IALILEPICC(Sar)ERAA-dPEG24 (SEQ ID NO: |
| | 32) |
| PA-R13Sar-dPEG24 | IALILEPICCQE(Sar)AA-dPEG24 (SEQ ID NO: |
| | 33) |
| PA-A14Sar-dPEG24 | IALILEPICCQER(Sar)A-dPEG24 (SEQ ID NO: |
| | 34) |
| E6SarP7Sar | IALIL(Sar)(Sar)ICCQERAA (SEQ ID NO: 35) |
| E6SarC9Sar | IALIL(Sar)PI(Sar)CQERAA (SEQ ID NO: 36) |
| Q11SarP7Sar | IALILE(Sar)ICC(Sar)ERAA (SEQ ID NO: 37) |
| Q11SarC9Sar | IALILEPI(Sar)C(Sar)ERAA (SEQ ID NO: 38) |
| R13SarP7Sar | IALILE(Sar)ICCQE(Sar)AA (SEQ ID NO: 39) |
| R13SarC9Sar | IALILEPI(Sar)CQE(Sar)AA (SEQ ID NO: 40) |
| A14SarP7Sar | IALILE(Sar)ICCQER(Sar)A (SEQ ID NO: 41) |
| A14SarC9Sar | IALILEPI(Sar)CQER(Sar)A (SEQ ID NO: 42) |
| E6AE12A-dPEG24 | IALILAPICCQARAA-dPEG24 (SEQ ID NO: 43) |
| E6AE12AC9Sar | IALILAPI(Sar)CQARAA (SEQ ID NO: 44) |
| E6AE12AP7Sar | IALILA(Sar)ICCQARAA (SEQ ID NO: 45) |

PIC1 variants may comprise peptides with at least one of the amino acids of the PA sequence deleted. PIC1 variants may comprise peptides with an amino acid inserted into the PA sequence. PIC1 variants may comprise peptides with at least one of the amino acids of the PA sequence substituted with another amino acid, such as alanine, a modified amino acid or an amino acid derivative, such as sarcosine (Sar).

In some embodiments, PIC1 comprises one or more PEG moieties. The PEG moieties may be attached to the N-terminus, the C-terminus, or both the N-terminus and C-terminus by PEGylation. In one or more embodiments, 24 PEG moieties are attached to the N-terminus. In one or more embodiments, 24 PEG moieties are attached to the C-terminus. In one or more embodiments, 24 PEG moieties are attached to the N-terminus and to the C-terminus. In one or more embodiments, 1, 2, 3, 4, 5, 6, 7, 8, 9, 10, 11, 12, 13, 14, 15, 16, 17, 18, 19, 20, 21, 22, 23, or 24 PEG moieties are attached to the N-terminus. In one or more embodiments, 1, 2, 3, 4, 5, 6, 7, 8, 9, 10, 11, 12, 13, 14, 15, 16, 17, 18, 19, 20, 21, 22, 23, or 24 PEG moieties are attached to the C-terminus. In one or more embodiments, 1, 2, 3, 4, 5, 6, 7, 8, 9, 10, 11, 12, 13, 14, 15, 16, 17, 18, 19, 20, 21, 22, 23, or 24 PEG moieties are attached to both the N-terminus and the C-terminus.

The PIC1 peptide may be a synthetic peptide. A synthetic peptide is prepared in vitro. Synthetic peptides can be prepared according to various methods known in the art. For example, a synthetic peptide can be prepared by sequentially coupling individual amino acids to form the peptide. In some embodiments, the carboxyl group of individual amino acids is sequentially coupled to the amino terminus of a growing peptide chain. Protecting groups can be used to prevent unwanted side reactions from occurring during the coupling process. Peptide synthesis can occur in liquid phase or in solid phase.

Exemplary PIC1 peptides include, but are not limited to, PA-dPEG24 (a peptide comprising the polar assortant (PA) sequence and 24 PEG moieties at the C-terminus), PA-dPEG20 (comprising 20 PEG moieties at the C-terminus), PA-dPEG16 (comprising 16 PEG moieties at the C-terminus), PA-dPEG12 (comprising 12 PEG moieties at the C-terminus), PA-dPEG08 (comprising 8 PEG moieties at the C-terminus), PA-dPEG06 (comprising 6 PEG moieties at the C-terminus), PA-dPEG04 (comprising 4 PEG moieties at the C-terminus), PA-dPEG03 (comprising 3 PEG moieties at the C-terminus), and PA-dPEG02 (comprising two PEG moieties at the C-terminus).

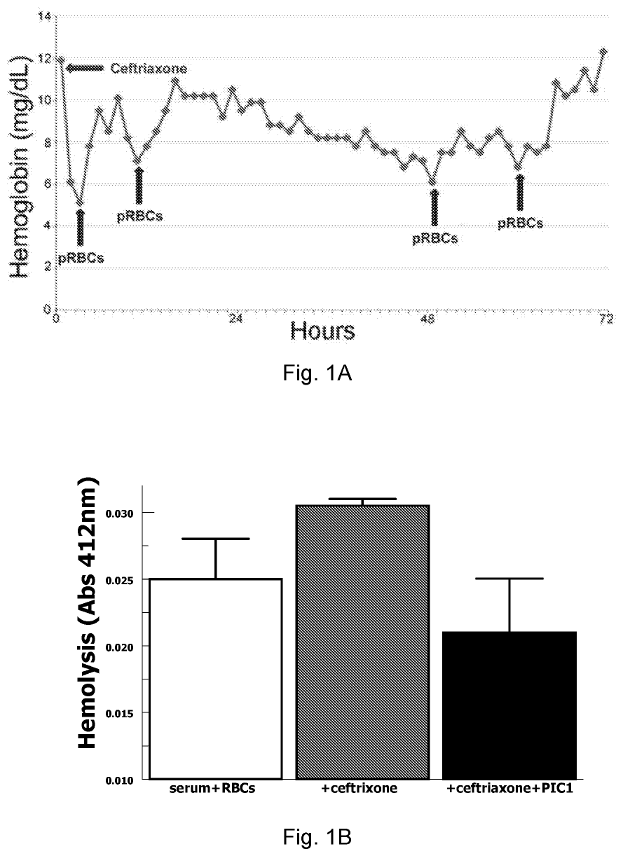

Experimental data provided herein shows that addition of PIC1 suppressed ceftriaxone-mediated hemolysis in a blood sample of a patient who underwent ceftriaxone-mediated hemolysis. The suppressive effect of PIC1 indicates that such hemolysis may be mediated by the classical complement pathway. These results suggest that classical complement pathway inhibitors can be used clinically to treat drug-induced hemolysis, e.g., CIIHA, and to prevent further hemolysis, particularly in a clinical setting in which there is a C3-positive DAT. The long half-life of ceftriaxone of 8 to 9 hours suggests that complement activation and hemolysis will continue to occur for a period of time as suggested by the tested patient's course (as shown FIG. 1A) and can be moderated by PIC1. Repeated administration of PIC1 and PIC1 variants may be performed until the drug, e.g., ceftriaxone, has sufficiently cleared the patient's system. Such repeated administration may occur over one, two, three, four, five, six, or even seven days.

In various embodiments, a therapeutically effective amount of the classical complement pathway inhibitor, e.g., PIC1 or a PIC1 variant, is administered. The therapeutically effective amount of the classical complement pathway inhibitor compound varies depending on several factors, such as the severity of the condition, the time of administration, the route of administration, the rate of excretion of the compound employed, the duration of treatment, the co-therapy involved, and the age, gender, weight, and condition of the subject, etc. One of ordinary skill in the art can determine the therapeutically effective amount. Accordingly, one of ordinary skill in the art may need to titer the dosage and modify the route of administration to obtain the maximal therapeutic effect.

In various embodiments, the classical complement pathway inhibitor, e.g., PIC1 or a PIC1 variant, is administered parenterally, e.g., such as by intravenous, intracerebral, intracerebroventricular, intramuscular, or subcutaneous injection, or by intravenous infusion. In various embodiments, PIC1 is administered once to the subject. PIC1 may be administered to the subject once a day, twice a day, three times a day, four times a day, five times a day, or six times a day. In various embodiments, the PIC1 is administered for one day, two days, three days, four days, five days, six days, or until the drug causing the hemolysis has underwent a period of one, two, three, four, five, six or seven half-lives in the patient. In various embodiments, the PIC1 variant is administered once to the subject. The PIC1 variant may be administered to the subject once a day, twice a day, three times a day, four times a day, five times a day, or six times a day.

The dosage or amount of the classical complement pathway inhibitor, e.g., PIC1 or a PIC1 variant, depends on the individual case and is, as is customary, to be adapted to the individual circumstances to achieve an optimum effect. The therapeutically effective amount can vary depending on several factors, such as the condition being treated, the severity of the condition, the time of administration, the route of administration, the rate of excretion of the compound employed, the duration of treatment, the co-therapy involved, and the age, gender, weight, and condition of the subject, etc. One of ordinary skill in the art can determine the therapeutically effective amount. Accordingly, one of ordinary skill in the art may need to titer the dosage and modify the route of administration to obtain the maximal therapeutic effect.

The effective daily dose of the classical complement pathway inhibitor, e.g., PIC1 or a PIC1 variant, generally is within the range of from 1 to 300 milligrams per kilogram (mg/kg) of body weight. In some embodiments, the daily dose is within the range of from 4 to 300 mg/kg. In some embodiments, the daily dose is within the range of from 10 to 125 mg/kg. In some embodiments, the daily dose is within the range of from 1 to 75 mg/kg. In some embodiments, the daily dose is within the range of from 30 to 100 mg/kg. In some embodiments, the daily dose is within the range of from 50 to 125 mg/kg. In some embodiments, the daily dose is within the range of from 50 to 100 mg/kg. In some embodiments, the daily dose is within the range of from 60 to 90 mg/kg. In some embodiments, the daily dose is within the range of from 70 to 100 mg/kg. In some embodiments, the daily dose is within the range of from 70 to 80 mg/kg.

In some embodiments, the daily dose is within the range of from 10 to 30 mg/kg. In some embodiments, the daily dose is within the range of from 20 to 40 mg/kg. In some embodiments, the daily dose is within the range of from 30 to 50 mg/kg. In some embodiments, the daily dose is within the range of from 40 to 60 mg/kg. In some embodiments, the daily dose is within the range of from 50 to 70 mg/kg. In some embodiments, the daily dose is within the range of from 60 to 80 mg/kg. In some embodiments, the daily dose is within the range of from 70 to 90 mg/kg.

In some embodiments, the daily dose is within the range of from about 80 to about 160 mg/kg. In some embodiments, the daily dose is within the range of from 80 to 160 mg/kg. In some embodiments, the daily dose is within the range of from 80 to 100 mg/kg. In some embodiments, the daily dose is within the range of from 90 to 110 mg/kg. In some embodiments, the daily dose is within the range of from 100 to 120 mg/kg. In some embodiments, the daily dose is within the range of from 110 to 130 mg/kg. In some embodiments, the daily dose is within the range of from 120 to 140 mg/kg. In some embodiments, the daily dose is within the range of from 130 to 150 mg/kg. In some embodiments, the daily dose is within the range of from 140 to 160 mg/kg.

In some embodiments, the daily dose is within the range of from about 150 to about 170 mg/kg. In some embodiments, the daily dose is within the range of from about 160 to about 180 mg/kg. In some embodiments, the daily dose is within the range of from about 170 to about 190 mg/kg. In some embodiments, the daily dose is within the range of from about 180 to about 300 mg/kg.

In various embodiments, the above doses can be achieved through a 1-6 time(s) daily dosing regimen. In some embodiments, the classical complement pathway inhibitor, e.g., PIC1 or a PIC1 variant, is administered once a day. In some embodiments, the classical complement pathway inhibitor is administered twice a day. In some embodiments, the classical complement pathway inhibitor is administered three times a day. In some embodiments, the classical complement pathway inhibitor is administered four times a day. In some embodiments, the classical complement pathway inhibitor is administered five times a day. In some embodiments, the classical complement pathway inhibitor is administered six times a day. Alternatively, optimal treatment can be achieved through a sustained release formulation with a less frequent dosing regimen.

The methods described herein may render the subject less dependent on transfusions or transfusion-independent. Administration of the classical complement pathway inhibitor, e.g., PIC1 or a PIC1 variant, may allow for the subject to recover without receiving a transfusion, or by receiving fewer transfusions as compared to a subject not administered the classical complement pathway inhibitor.

In various embodiments, the classical complement pathway inhibitor is administered with a blood transfusion, before a blood transfusion, or after a blood transfusion. The blood transfusion may be a packed red blood cell transfusion.

In various embodiments, the methods described herein are effective to reduce the duration of time the patient suffers from hemolysis by at least 25%, at least 30%, at least 35%, at least 40%, at least 45%, at least 50%, at least 55%, at least 60%, at least 65%, at least 70%, at least 75%, at least 80%, as compared to a patient who was not treated with the classical complement pathway inhibitor. The methods may also provide for a rapid reduction in hemoglobinuria.

Hemolysis may be assayed by measuring the lactate dehydrogenase (LDH) levels in the patient's bloodstream. LDH catalyzes the interconversion of pyruvate and lactate. Red blood cells metabolize glucose to lactate, which is released into the blood and is taken up by the liver. LDH levels are used as an objective indicator of hemolysis. As those skilled in the art will appreciate, measurements of “upper limit of normal” levels of LDH will vary from lab to lab depending on a number of factors including the particular assay employed and the precise manner in which the assay is conducted. In various embodiments, the methods described herein can reduce hemolysis in a patient afflicted with a hemolytic disease as reflected by a reduction of LDH levels in the patients to within 20% of the upper limit of normal LDH levels. In various alternative embodiments, the methods described herein can reduce LDH levels by at least 40%, 45%, 50%, 55%, 60%, 65%, 70%, 75%, 80%, 85%, or 90% of the LDH level before the classical complement pathway inhibitor was administered to the subject.

The methods described herein may be effective to alleviate one or more of the following symptoms associated with hemolysis: abdominal pain, fatigue, dysphagia, thrombosis, dyspnea, insomnia. Symptoms can be the direct result of lysis of red blood cells (e.g., hemoglobinuria, anemia, fatigue, and low red blood cell count) or the symptoms can result from low nitric oxide (NO) levels in the patient's bloodstream (e.g., abdominal pain, dysphagia, and thrombosis). Abdominal pain may arise from the inability of a patient's natural levels of haptoglobin to process all the free hemoglobin released into the bloodstream as a result of intravascular hemolysis, resulting in the scavenging of NO and intestinal dystonia and spasms. Dysphagia may result from the inability of a patient's natural levels of haptoglobin to process all the free hemoglobin released into the bloodstream as a result of intravascular hemolysis, resulting in the scavenging of NO and esophageal spasms. Hemoglobinuria may result from the inability of a patient's natural levels of haptoglobin to process all the free hemoglobin released into the bloodstream as a result of intravascular hemolysis. Hemoglobinuria may be associated with red, brown, or darker urine. Hemoglobinuria can cause acute kidney injury. Hemoglobinuria may resolve rapidly after the classical complement pathway inhibitor is administered. Thrombosis is believed to be associated with scavenging of NO by free hemoglobin released into the bloodstream as a result of intravascular hemolysis.

In various embodiments, additional compounds are administered to the subject to increase hematopoiesis. These additional compounds may be administered concurrently with the classical complement pathway inhibitor, or after administration of the classical complement pathway inhibitor. The additional compounds may even be administered 1, 2, 3, 4, 5, 6 or 7 days after discontinuation of the classical complement pathway inhibitor. Exemplary hematopoiesis-increasing compounds include, but are not limited to, steroids, immunosuppressants (e.g., cyclosporin), anti-coagulants (e.g., warfarin), folic acid, iron, erythropoietin (EPO) and antithymocyte globulin (ATG), antilymphocyte globulin (ALG), EPO derivatives, and darbepoetin alfa.

The classical complement pathway inhibitor, e.g., PIC1 or a PIC1 variant, may be formulated into pharmaceutical compositions or formulations with one or more pharmaceutically acceptable carriers/excipients. The pharmaceutical compositions may comprise one or more of the PIC1 and PIC1 variants disclosed herein. The pharmaceutical compositions may also further comprise one or more other pharmaceutically or biologically active ingredients as defined above. Pharmaceutical formulations comprising PIC1 and PIC1 variants preferably meet sterility, pyrogenicity, general safety, and purity standards, as required by the offices of the Food and Drug Administration (FDA).

In various embodiments, the pharmaceutically acceptable carrier or excipient is compatible with the other ingredients of a pharmaceutical composition and not deleterious to the recipient thereof. Exemplary carriers and excipients include, but are not limited to, solvents, diluents, buffers (e.g., neutral buffered saline, phosphate buffered saline, or optionally Tris-HCl, acetate or phosphate buffers), solubilizers (e.g., Tween 80 or Polysorbate 80), colloids, dispersion media, vehicles, fillers, chelating agents (e.g., EDTA or glutathione), amino acids (e.g., glycine), proteins, disintegrants, binders, lubricants, wetting agents, stabilizers, emulsifiers, sweeteners, colorants, flavorings, aromatizers, thickeners, agents for achieving a depot effect, coatings, antifungal agents, preservatives (e.g., Thimerosal™ or benzyl alcohol), antioxidants (e.g., ascorbic acid or sodium metabisulfite), tonicity controlling agents, absorption delaying agents, adjuvants, bulking agents (e.g., lactose or mannitol) and the like.

For example, the pharmaceutical composition may be formulated for parenteral administration. For example, the composition may be formulated for administration by one or more of the following means: intravenous, intracerebral, intracerebroventricular, intramuscular, or subcutaneous injection, or intravenous infusion. The parenterally acceptable aqueous solution may be pyrogen-free and have suitable pH, isotonicity and stability.

For subcutaneous or intravenous administration, the pharmaceutical composition may comprise one or more of solubilizers, emulsifiers, and further auxiliaries that are brought into solution, suspension, or emulsion. The active ingredient(s) can also be lyophilized and the lyophilizates obtained used, for example, for the production of injection or infusion preparations. Suitable solvents are, for example, water, physiological saline solution, or alcohols (e.g., ethanol, propanol or glycerol), sugar solutions such as glucose or mannitol solutions, or alternatively mixtures of the various solvents mentioned. The injectable solutions or suspensions may be formulated using suitable non-toxic, parenterally-acceptable diluents, or solvents, such as mannitol, 1,3-butanediol, water, Ringer's solution, or isotonic sodium chloride solution, or suitable dispersing or wetting and suspending agents, such as sterile, bland, fixed oils, including synthetic mono- or diglycerides, and fatty acids, including oleic acid.

Alternatively, the pharmaceutical composition may be formulated for oral administration, such as for follow up dosing of a classical complement pathway inhibitor, e.g., PIC1 or a PIC1 variant. Exemplary oral administration dosage forms include, but are not limited to, pills, tablets, lacquered tablets, sugar-coated tablets, granules, hard and soft gelatin capsules, aqueous, alcoholic or oily solutions, syrups, emulsions or suspensions. Other dosage forms include, for example, ointments, tinctures, sprays or transdermal therapeutic systems (e.g., a skin patch), or by inhalation in the form of nasal sprays or aerosol mixtures, or, for example, in the form of microcapsules, implants or rods.

Oral administration of a pharmaceutical composition comprising at least one classical complement pathway inhibitor compound as disclosed herein, may be performed by uniformly and intimately blending together a suitable amount of said component in the form of a powder, optionally also including a finely divided solid carrier, and encapsulating the blend in, for example, a hard gelatin capsule. The solid carrier can include one or more substances, which act as binders, lubricants, disintegrating agents, coloring agents, and the like. Suitable solid carriers include, for example, calcium phosphate, magnesium stearate, talc, sugars, lactose, dextrin, starch, gelatin, cellulose, polyvinylpyrrolidine, low melting waxes and ion exchange resins.

The pharmaceutical compositions may be formulated so as to provide rapid, sustained, or delayed release of the active ingredient(s) contained therein, for example using micelles, liposomes or hydrophilic polymeric matrices based on natural gels or synthetic polymers.

Pharmaceutical formulations may be adapted for administration by any appropriate route, for example, by the oral, nasal, topical (including buccal, sublingual, or transdermal), or parenteral (including subcutaneous, intracutaneous, intramuscular, intraarticular, intraperitoneal, intrasynovial, intrasternal, intrathecal, intralesional, intravenous, or intradermal injections or infusions) route. For human administration, the formulations preferably meet sterility, pyrogenicity, general safety, and purity standards, as required by the offices of the Food and Drug Administration (FDA).

In another aspect is provided an assay for determining if the drug-induced hemolytic anemia is treatable by a complement-mediated pathway inhibitor. The assay may comprise taking a blood sample from the patient's body. The assay comprises treating a portion of the blood sample with a complement-mediated pathway inhibitor (e.g., PIC1 or a PIC1 variant). Another portion of the blood sample is not treated with the inhibitor. The assay then comprises measuring the extent of hemolysis in both (i) the portion of the blood sample treated with the complement-mediated pathway inhibitor and (ii) the untreated blood sample. If the hemolysis is reduced in the treated portion of the blood sample as compared to the untreated blood sample, then the hemolytic anemia is determined to be treatable with a complement-mediated pathway inhibitor (e.g., PIC1 or a PIC1 variant). The assay may further comprise treating a second portion of the blood sample with both the complement-mediated pathway inhibitor and the drug thought to induce the drug-induced hemolytic anemia.

### EXAMPLES

The present invention is also described and demonstrated by way of the following example. However, the use of this and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Example 1

A 10-year old female patient with chronic active Epstein-Barr virus disease and hemophagocytic lymphohistiocytosis presented with ceftriaxone-induced hemolytic anemia. The patient had been evaluated in the emergency department for fever and possible sepsis after recently receiving chemotherapy. In the emergency department she received a dose of ceftriaxone (50 mg/kg). The patient had received ceftriaxone on three previous occasions with no history of adverse reaction. Within an hour she developed back pain, tachycardia and tachypnea. Over the next three hours she developed worsening distress, failed continuous positive airway pressure and required endotracheal intubation with mechanical ventilation. She also experienced hypotension requiring fluid resuscitation and a continuous epinephrine infusion.

Prior to receiving ceftriaxone she had an erythrocyte hemoglobin level of 11.9 g/dL. Four hours later, her hemoglobin had decreased to 6.1 g/dL, followed by a point-of-care hemoglobin of 5.1 g/dL. She received four packed red blood cell transfusions (each 10 mL/kg) within 72 hours, after which, her hemoglobin stabilized at her initial baseline (FIG. 1A). Urinalysis demonstrated hemoglobinuria and bilirubinuria. She had a positive direct antiglobulin test (DAT) for both IgG and C3. There were spherocytes on her peripheral blood smear as well as red blood cell aggregation. On admission, one day later, and five days later, her total bilirubin level was 1.5 mg/dL, 10.7 mg/dL, and 23.1 mg/dL respectively, with 90% being conjugated. On admission, her LDH was 514 U/L and increased to 42,093 U/L two days later. The patient's renal function declined 24 hours after ceftriaxone, with her BUN doubling from 12 mg/dL to 25 mg/dL and serum creatinine tripling to 0.9 mg/dL from 0.3 mg/dL. She continued to require ionotropic blood pressure support for three days and required mechanical ventilation for 16 days.

The American Red Cross tested a blood sample from the day after admission and reported strong reactivity at 37° C., with the DAT result indicating a strong likelihood that the patient has antibody to ceftriaxone.

To confirm that the patient experienced ceftriaxone-induced complement-mediated hemolysis, the Complement Hemolysis Using Human Erythrocytes (CHUHE) assay was modified such that ceftriaxone was added to the patient's serum to enhance lysis of the erythrocytes. The data provided below shows that ceftriaxone initiated a classical complement pathway-mediated hemolysis by in vitro reversal with Peptide Inhibitor of Complement C1 (PIC1).

The patient's blood and sera were provided as discarded de-identified samples from residual specimens in the Blood Bank. PA-dPEG24, a PIC1 peptide, was synthesized by PolyPeptide Group (San Diego, Calif.). Standard veronal complement buffers were utilized.

The modified CHUHE assay was performed as follows. The patient's sera (0.1 ml) was combined with ceftriaxone (10 μg/ml final concentration) in an ice-water bath for 30 minutes to enhance immune complex formation. This solution was then warmed to 24° C. 5×10⁷ of the patient's erythrocytes were added, with or without PIC1 (final concentration 0.75 mM). Samples were incubated at 37° C. for one hour, with hemolysis stopped by the addition of 2.0 ml of GVBS-EDTA buffer (veronal-buffered saline with 0.1% gelatin and 10 mM EDTA). Erythrocytes were sedimented, and free hemoglobin was measured by spectrophotometry at 412 nm. Two independent experiments (n=2) were performed. Without wishing to be bound by theory, adaptation of the CHUHE hemolytic assay by adding exogenous ceftriaxone to the patient's serum and erythrocytes allowed for evaluation as to whether the hemolysis was ceftriaxone-initiated.

The experiments showed that adding ceftriaxone (10 μg/ml) increased complement-mediated hemolysis (p=0.02) of her erythrocytes by her serum (FIG. 1B). Hemolysis was enhanced in the presence of ceftriaxone, confirming that ceftriaxone was the initiating stimulus. Addition of PIC1 was also performed to evaluate whether hemolysis occurred via the classical complement pathway. Adding PIC1 (0.75 mM) and ceftriaxone together to the patient's serum and erythrocytes decreased hemolysis, as compared with adding ceftriaxone alone (p=0.03). These results demonstrated that the patient's hemolysis was mediated by the classical complement pathway. Addition of both 0.75 mM PIC1 and ceftriaxone also returned hemolysis to the level of the no ceftriaxone control. Together, these tests confirmed that the hemolysis was initiated by ceftriaxone and mediated via the classical complement pathway.

### Example 2

A 12 year-old patient is tested for possible hemolysis as a result of treatment with ceftriaxone. The patient has an erythrocyte hemoglobin level of 6.5 g/dL, a total bilirubin level of 12.4 mg/dL, and an LDH of 16,500. The patient receives packed red blood cell transfusion, and ceftriaxone is discontinued.

A blood sample is taken from the patient. The modified CHUHE assay described in Example 1 is performed. As compared to the control, hemolysis increased by 25% in the sample with 10 μg/ml ceftriaxone added. Hemolysis decreased by 10% where both 10 μg/ml ceftriaxone and 0.75 M PIC1 are added, as compared to the control. The results indicate that the patient does have ceftriaxone-induced hemolysis and that the patient could benefit from PIC1 treatment.

A dose of 150 mg/kg PIC1 is promptly administered to the patient. Four hours later, the patient has an erythrocyte hemoglobin level of 7.9 g/dL, a total bilirubin level of 8.4 mg/dL, and an LDH of 10,500, all of which show improvement. Over the following three days, a daily dose of 150 mg/kg PIC1 is administered to the patient. The bilirubin and LDH levels continue to improve. The patient receives additional packed red blood cell transfusions as needed. The patient is later found to have completely recovered from the hemolysis.

The present application describes a number of examples and embodiments of the invention. Nevertheless, it must be borne in mind that various modifications of the described examples and embodiments can be developed, while not departing from the scope and the essence of the invention in principle. With this in mind, other embodiments are included in the scope of the items listed below. At that, all the numerical ranges described herein include all the sub ranges contained therein, as well as any individual values within the scope of these ranges. All publications, patents and patent applications mentioned in this description are hereby incorporated by reference.

1. A method of treating hemolytic anemia in a subject comprising administering a classical complement pathway inhibitor to the subject.

2. The method of claim 1, wherein the hemolytic anemia is induced by a drug.

3. The method of claim 2, wherein the drug is an antibiotic.

4. The method of claim 3, wherein the antibiotic is cefotetan, ceftriaxone, or piperacillin.

5. The method of claim 4, wherein the drug is ceftriaxone.

6. The method of claim 1, wherein the classical complement pathway inhibitor is PIC1.

7. The method of claim 6, wherein the PIC1 comprises one or more PEG moieties.

8. The method of claim 6, wherein the PIC1 comprises at least about 90% sequence identity to at least one amino acid sequence selected from the group consisting of SEQ ID NO: 3-47.

9. The method of claim 6, wherein the PIC1 is PA-dPEG24.

10. The method of claim 9, wherein the PA-dPEG24 comprises the sequence of
(SEQ ID NO: 19)
IALILEPICCQERAA-dPEG24.

11. The method of claim 1, wherein the classical complement pathway inhibitor is administered once per day.

12. The method of claim 1, wherein the classical complement pathway inhibitor is administered two times per day.

13. The method of claim 1, wherein the classical complement pathway inhibitor is administered for 1, 2, 3, 4, 5, or 6 days.


SEQUENCE LISTING (45 sequences)

SEQ ID NO: 1 - protein, 15 aa, Artificial Sequence
  Description of Artificial Sequence Synthetic peptide
IALILEPICCQERAA

SEQ ID NO: 2 - protein, 15 aa, Artificial Sequence
  Description of Artificial Sequence Synthetic peptide
  MOD_RES (1)..(1): Sarcosine
XALILEPICCQERAA

SEQ ID NO: 3 - protein, 15 aa, Artificial Sequence
  Description of Artificial Sequence Synthetic peptide
  MOD_RES (2)..(2): Sarcosine
IXLILEPICCQERAA

SEQ ID NO: 4 - protein, 15 aa, Artificial Sequence
  Description of Artificial Sequence Synthetic peptide
  MOD_RES (3)..(3): Sarcosine
IAXILEPICCQERAA

SEQ ID NO: 5 - protein, 15 aa, Artificial Sequence
  Description of Artificial Sequence Synthetic peptide
  MOD_RES (4)..(4): Sarcosine
IALXLEPICCQERAA

SEQ ID NO: 6 - protein, 15 aa, Artificial Sequence
  Description of Artificial Sequence Synthetic peptide
  MOD_RES (5)..(5): Sarcosine
IALIXEPICCQERAA

SEQ ID NO: 7 - protein, 15 aa, Artificial Sequence
  Description of Artificial Sequence Synthetic peptide
  MOD_RES (6)..(6): Sarcosine
IALILXPICCQERAA

SEQ ID NO: 8 - protein, 15 aa, Artificial Sequence
  Description of Artificial Sequence Synthetic peptide
  MOD_RES (7)..(7): Sarcosine
IALILEXICCQERAA

SEQ ID NO: 9 - protein, 15 aa, Artificial Sequence
  Description of Artificial Sequence Synthetic peptide
  MOD_RES (8)..(8): Sarcosine
IALILEPXCCQERAA

SEQ ID NO: 10 - protein, 15 aa, Artificial Sequence
  Description of Artificial Sequence Synthetic peptide
  MOD_RES (9)..(9): Sarcosine
IALILEPIXCQERAA

SEQ ID NO: 11 - protein, 15 aa, Artificial Sequence
  Description of Artificial Sequence Synthetic peptide
  MOD_RES (10)..(10): Sarcosine
IALILEPICXQERAA

SEQ ID NO: 12 - protein, 14 aa, Artificial Sequence
  Description of Artificial Sequence Synthetic peptide
  MOD_RES (10)..(10): Sarcosine
ALILEPICCXERAA

SEQ ID NO: 13 - protein, 15 aa, Artificial Sequence
  Description of Artificial Sequence Synthetic peptide
  MOD_RES (12)..(12): Sarcosine
IALILEPICCQXRAA

SEQ ID NO: 14 - protein, 15 aa, Artificial Sequence
  Description of Artificial Sequence Synthetic peptide
  MOD_RES (13)..(13): Sarcosine
IALILEPICCQEXAA

SEQ ID NO: 15 - protein, 15 aa, Artificial Sequence
  Description of Artificial Sequence Synthetic peptide
  MOD_RES (14)..(14): Sarcosine
IALILEPICCQERXA

SEQ ID NO: 16 - protein, 15 aa, Artificial Sequence
  Description of Artificial Sequence Synthetic peptide
  MOD_RES (15)..(15): Sarcosine
IALILEPICCQERAX

SEQ ID NO: 17 - protein, 15 aa, Artificial Sequence
  Description of Artificial Sequence Synthetic peptide
  misc_feature (1)..(1): N-term dPEG24
  misc_feature (15)..(15): C-term dPEG24
IALILEPICCQERAA

SEQ ID NO: 18 - protein, 15 aa, Artificial Sequence
  Description of Artificial Sequence Synthetic peptide
  misc_feature (1)..(1): N-term dPEG24
IALILEPICCQERAA

SEQ ID NO: 19 - protein, 15 aa, Artificial Sequence
  Description of Artificial Sequence Synthetic peptide
  misc_feature (15)..(15): C-term dPEG24
IALILEPICCQERAA

SEQ ID NO: 20 - protein, 15 aa, Artificial Sequence
  Description of Artificial Sequence Synthetic peptide
  misc_feature (15)..(15): C-term dPEG20
IALILEPICCQERAA

SEQ ID NO: 21 - protein, 15 aa, Artificial Sequence
  Description of Artificial Sequence Synthetic peptide
  misc_feature (15)..(15): C-term dPEG16
IALILEPICCQERAA

SEQ ID NO: 22 - protein, 15 aa, Artificial Sequence
  Description of Artificial Sequence Synthetic peptide
  misc_feature (15)..(15): C-term dPEG12
IALILEPICCQERAA

SEQ ID NO: 23 - protein, 15 aa, Artificial Sequence
  Description of Artificial Sequence Synthetic peptide
  misc_feature (15)..(15): C-term dPEG08
IALILEPICCQERAA

SEQ ID NO: 24 - protein, 15 aa, Artificial Sequence
  Description of Artificial Sequence Synthetic peptide
  misc_feature (15)..(15): C-term dPEG06
IALILEPICCQERAA

SEQ ID NO: 25 - protein, 15 aa, Artificial Sequence
  Description of Artificial Sequence Synthetic peptide
  misc_feature (15)..(15): C-term dPEG04
IALILEPICCQERAA

SEQ ID NO: 26 - protein, 15 aa, Artificial Sequence
  Description of Artificial Sequence Synthetic peptide
  misc_feature (15)..(15): C-term dPEG03
IALILEPICCQERAA

SEQ ID NO: 27 - protein, 15 aa, Artificial Sequence
  Description of Artificial Sequence Synthetic peptide
  misc_feature (15)..(15): C-term dPEG02
IALILEPICCQERAA

SEQ ID NO: 28 - protein, 15 aa, Artificial Sequence
  Description of Artificial Sequence Synthetic peptide
  MOD_RES (9)..(9): Sarcosine
IALILEPIXAQERAA

SEQ ID NO: 29 - protein, 14 aa, Artificial Sequence
  Description of Artificial Sequence Synthetic peptide
  MOD_RES (9)..(9): Sarcosine
IALILEPIXQERAA

SEQ ID NO: 30 - protein, 15 aa, Artificial Sequence
  Description of Artificial Sequence Synthetic peptide
  MOD_RES (7)..(7): Sarcosine
  MOD_RES (9)..(9): Sarcosine
IALILEXIXCQERAA

SEQ ID NO: 31 - protein, 15 aa, Artificial Sequence
  Description of Artificial Sequence Synthetic peptide
  MOD_RES (6)..(6): Sarcosine
  misc_feature (15)..(15): C-term dPEG24
IALILXPICCQERAA

SEQ ID NO: 32 - protein, 15 aa, Artificial Sequence
  Description of Artificial Sequence Synthetic peptide
  MOD_RES (11)..(11): Sarcosine
  misc_feature (15)..(15): C-term dPEG24
IALILEPICCXERAA

SEQ ID NO: 33 - protein, 15 aa, Artificial Sequence
  Description of Artificial Sequence Synthetic peptide
  MOD_RES (13)..(13): Sarcosine
  misc_feature (15)..(15): C-term dPEG24
IALILEPICCQEXAA

SEQ ID NO: 34 - protein, 15 aa, Artificial Sequence
  Description of Artificial Sequence Synthetic peptide
  MOD_RES (14)..(14): Sarcosine
  misc_feature (15)..(15): C-term dPEG24
IALILEPICCQERXA

SEQ ID NO: 35 - protein, 15 aa, Artificial Sequence
  Description of Artificial Sequence Synthetic peptide
  MOD_RES (6)..(7): Sarcosine
IALILXXICCQERAA

SEQ ID NO: 36 - protein, 15 aa, Artificial Sequence
  Description of Artificial Sequence Synthetic peptide
  MOD_RES (6)..(6): Sarcosine
  MOD_RES (9)..(9): Sarcosine
IALILXPIXCQERAA

SEQ ID NO: 37 - protein, 15 aa, Artificial Sequence
  Description of Artificial Sequence Synthetic peptide
  MOD_RES (7)..(7): Sarcosine
  MOD_RES (11)..(11): Sarcosine
IALILEXICCXERAA

SEQ ID NO: 38 - protein, 15 aa, Artificial Sequence
  Description of Artificial Sequence Synthetic peptide
  MOD_RES (9)..(9): Sarcosine
  MOD_RES (11)..(11): Sarcosine
IALILEPIXCXERAA

SEQ ID NO: 39 - protein, 15 aa, Artificial Sequence
  Description of Artificial Sequence Synthetic peptide
  MOD_RES (7)..(7): Sarcosine
  MOD_RES (13)..(13): Sarcosine
IALILEXICCQEXAA

SEQ ID NO: 40 - protein, 15 aa, Artificial Sequence
  Description of Artificial Sequence Synthetic peptide
  MOD_RES (9)..(9): Sarcosine
  MOD_RES (13)..(13): Sarcosine
IALILEPIXCQEXAA

SEQ ID NO: 41 - protein, 15 aa, Artificial Sequence
  Description of Artificial Sequence Synthetic peptide
  MOD_RES (7)..(7): Sarcosine
  MOD_RES (14)..(14): Sarcosine
IALILEXICCQERXA

SEQ ID NO: 42 - protein, 15 aa, Artificial Sequence
  Description of Artificial Sequence Synthetic peptide
  MOD_RES (9)..(9): Sarcosine
  MOD_RES (14)..(14): Sarcosine
IALILEPIXCQERXA

SEQ ID NO: 43 - protein, 15 aa, Artificial Sequence
  Description of Artificial Sequence Synthetic peptide
  misc_feature (15)..(15): C-term dPEG24
IALILAPICCQARAA

SEQ ID NO: 44 - protein, 15 aa, Artificial Sequence
  Description of Artificial Sequence Synthetic peptide
  MOD_RES (9)..(9): Sarcosine
IALILAPIXCQARAA

SEQ ID NO: 45 - protein, 15 aa, Artificial Sequence
  Description of Artificial Sequence Synthetic peptide
  MOD_RES (7)..(7): Sarcosine
IALILAXICCQARAA